(19) United States
(12) Patent Application Publication
(10) Pub. No.: US 2010/0004645 A1
(43) Pub. Date: Jan. 7, 2010

(54) INVASIVE DUAL-WAVELENGTH LASER ACUPUNCTURE

(75) Inventors: Sungho Jeong, Gwangju (KR); Kwang Hwan Oh, Gwangju (KR); Seok Hee Lee, Gwangju (KR); Chang Su Na, Gwangju (KR)

Correspondence Address: AMPACC Law Group, 3500 188th Street S.W., SUITE 103, Lynnwood, WA, 98037, US

(73) Assignee: Gwangju Institute of Science and Technology, Gwangju (KR)

(21) Appl. No.: 12/494,207

(22) Filed: Jun. 29, 2009

(30) Foreign Application Priority Data
Jul. 1, 2008 (KR) 10-2008-0063437

(51) Int. Cl. A61B 18/22

(52) U.S. Cl. 606/16

(57) ABSTRACT

An invasive laser acupuncture includes a first semiconductor laser connected to a first optical fiber acupuncture and providing red-based laser beam with the first optical fiber acupuncture; a second semiconductor laser connected to a second optical fiber acupuncture and providing a green-based laser beam with the second optical fiber acupuncture; and a driving circuit independently driving the first semiconductor laser and the second semiconductor laser in a continuous mode or a pulse mode by a switching operation. Since red and green lasers can independently be driven in the continuous mode and the pulse mode, the red and green laser can easily be adopted in a reinforcing and reducing treatment method in traditional oriental medicine. In addition, by using a metal-coated optical fiber acupuncture, the optical fiber acupuncture is injected directly into meridian pathways provided under an epidermal layer, such that it is possible to efficiently transmit a laser beam without loss.

### BACKGROUND OF THE INVENTION

1. Field of the Invention

The present invention relates to an invasive dual-wavelength laser acupuncture, and more particularly, to an invasive dual-wavelength laser acupuncture adoptable in a reinforcing and reducing treatment method.

2. Description of the Related Art

Phototherapy, which has been actively used in traditional oriental medicine in recent years treats diseases by stimulating meridian pathways and facilitating flow of vitality and blood with ray-based apparatuses or equipments, and uses natural or artificial rays. The phototherapy principally uses ultraviolet rays, visible rays, infrared rays, a laser, etc. When the rays are irradiated to affected parts to stimulate the meridian pathways, the meridian pathways are generally adjusted and treated. The treatment using the laser is one of the phototherapy. The laser is divided into a high-power laser and a high-power laser. Since the high-power laser destructs and evaporates cells within several seconds and removes a lesion without bleeding, edema, or damage of surrounding tissues during an operation, the high-power laser has been widely adopted in a surgical field, etc. such as the operation. Contrary to this, the low-power laser is usefully used as the phototherapy that induces the photosynthesis of organisms to grow up organisms and provides energy that is the source of a life.

The phototherapy using a He—Ne laser which is representative of the low-power laser was developed by Javan, etc. in the 1960s and began use in clinical medicine in Russia (the Soviet Union) in the 1970s. In 1980, after ultraviolet blood irradiation and oxygenation based on tumor research was developed by the Soviet Academy of Science, the phototherapy for the blood has been attempted while an influence on lymphocyte of the He—Ne laser has been researched. In 1990, the low level laser therapy (LLLT) was first developed by a team lead by a Wang Cheol Dan professor in China and has been also referred to as the intravascular laser irradiation on blood. As such, although a He—Ne laser having a wavelength of 633 nm has been principally used in known low-power laser acupuncture treatment for various diseases in the traditional oriental medicine, the wavelength of the laser has gradually diversified so as to be utilized in the clinical treatments, thus a semiconductor laser or a laser diode which can oscillate in various wavelength domains has rapidly developed in the semiconductor industry in recent years.

FIG. 1 is a block diagram illustrating laser acupuncture using a known He—Ne laser.

Referring to FIG. 1, a laser beam oscillated from the He—Ne laser 100 is focused through a focusing lens 110 and the position of the laser beam is controlled by using an optical alignment device 120 in order to efficiently inject the laser beam into an optical fiber 150.

The laser beam injected into the optical fiber 150 is transferred to an affected part of a treatment acceptor without causing loss. The laser beam transferred through the optical fiber 150 disperses with a divergence angle at an end of the optical fiber 150. Since this type of beam reduces the effect of the acupuncture, the laser beam is again focused through the focusing lens 130 before the laser beam is irradiated to the affected part and irradiated to a desired part.

When the optical fiber 150 is not used in the laser acupuncture using the known He—Ne laser 100, the laser beam oscillated from the He—Ne laser 100 is transferred to the affected part through a beam guide 151 attached with a plurality of reflection mirrors and focused through the focusing lens 130 before being irradiated to the affected part like the laser using the optical fiber 150.

Subsequently, the laser acupuncture using the known He—Ne laser essentially requires the focusing lens 110 for focusing a laser beam and the optical alignment device 120. In the case of removing the focusing lens 110 and the optical alignment device 120, since the beam guide 151 having a complicated structure is required as an optical transmission means, the treatment is inconvenient and frequent optical alignment is required.

As a method for solving the above-mentioned problems, the semiconductor laser or the laser diode that can oscillate in various wavelength domains has been developed to be used in the laser acupuncture with rapid growth of the semiconductor industry and as a result, the laser wavelength which can be utilized in the clinical treatment has been gradually extended.

In the related art, most of laser acupunctures that were developed in an early stage are based on the He—Ne laser having a wavelength of 633 nm, but laser acupuncture devices having a single wavelength, which uses a red or infrared ray semiconductor laser as a light source have been principally developed in recent years in order to achieve small-size and light-weighted laser acupuncture devices. Currently commercialized semiconductor lasers have wide oscillation domains ranging from 400 nm to 1550 nm and low power ranging from a several-mW level to high power of a several hundreds of W-level. The semiconductor laser that is principally adopted in the laser acupuncture is a red laser having a wavelength of 630 nm or more or a near infrared-rays laser having a wavelength band of 800 nm. The lasers having the wavelengths are principally adopted in warm-blooded treatment of the meridian pathways in the traditional oriental medicine.

FIG. 2 is a block diagram illustrating laser acupuncture using a known semiconductor laser and FIG. 3 is a circuit diagram illustrating a driving circuit for driving a known semiconductor laser.

Referring to FIG. 2, the laser beam oscillated from a semiconductor laser 160 attached with the optical fiber can be directly transmitted to the affected part through the optical fiber 150 and irradiated to the affected part after being focused through the focusing lens 130 in order to maximize the effect of the laser acupuncture like using the He—Ne laser 100. However, current must be supplied in order to drive the semiconductor laser 160.

Referring to FIG. 3, a current supply circuit for driving the known semiconductor laser 160 uses an LED driving circuit 170 in which a power supply Vs and one resistor R are connected to each other in series. Since the known driving circuit for driving the semiconductor laser uses a circuit for driving a light emitting diode (LED) constituted by connecting a power supply and a serial resistor to each other in order to simplify a system and save an element cost, overcurrent flows on the driving current to shorten the life-span of the semiconductor laser 160 and it is impossible to ensure stable laser power.

Further, in the known laser acupuncture, in driving a semiconductor laser pulse, it is difficult to arbitrarily adjust a pulse width, a pulse repetition rate, and a pulse peak value and in the case of using semiconductor lasers having various wavelengths, since one power supply system was used without an additional current supply device for oscillating each laser, it is difficult for the semiconductor lasers having different oscillation conditions to oscillate at the maximum power of each laser.

In the case of the laser acupuncture that was developed in the early stage, the treatment is performed by irradiating the beam oscillated from the laser directly to the affected part or irradiating the laser beam after focusing light by using the focusing lens. However, in recent years, many devices using the optical fiber for optical transmission have been developed and adopt a treatment method in which the laser beam is just irradiated to the affected part without causing any loss after being focused on the optical fiber. In particular, by this structure, since the laser beam is transferred into the optical fiber, the treatment acceptor does not need to be adjacent to a laser acupuncture treatment apparatus and the laser beam can arbitrarily be irradiated to the affected part of the treatment acceptor. However, most of the known laser acupuncture treatment apparatuses that have been developed up to now are a non-invasive type in which the laser beam is irradiated directly to the surface of a skin and injected into the meridian pathways and since the known laser acupuncture treatment apparatuses do not use a physical metal acupuncture without a pin and bleeding and can alleviate a worry about infection of other diseases, thus giving convenience and comfort to patients. For this reason, even in a domestic country, although many acupuncture treatment apparatuses and acupuncture treatment methods using the low-power laser are introduced and utilized, a report for verifying the effects is yet insufficient.

In the non-invasive laser acupuncture treatment apparatus, since the irradiated laser beam cannot efficiently reach the meridian pathways due to loss caused by reflection and scattering on the surface of the skin, the effect of the acupuncture cannot be normally achieved.

A correlation between the intensity of the irradiated laser beam and the intensity of the laser beam irradiated into the skin can be expressed by Equation 1.

I=Io×(1−ρ)×exp(−α×L) Equation 1

where, I represents the intensity of an injected laser beam, Io represents the intensity of an irradiated laser beam, ρ represents reflectance on the surface of a skin, α represents absorptance of the laser beam, and L represents an injection depth.

In general, the power of the laser for the laser acupuncture treatment is 5 mW or more and in the case of a human body, ρ=0.42 and α=0.3 mm−1. Further, in the metal acupuncture, since an injection depth is 20 mm or more from the surface of the skin, the power of the irradiated laser beam must be several Ws in consideration of the laser having the wavelength of 650 nm. However, since the skin of the human body can be damaged when the power of the laser is approximately at 0.5 W or more, it is difficult to expect a high treatment effect with respect to the non-invasive laser acupuncture which can only use low laser power.

In order to overcome the difficulty, the effect of the invasive laser acupuncture may be achieved by inserting and fixing the optical fiber into an intravenous injector. However, in the case of the intravenous injector, since the end of the injector is processed at an angle of 10 to 30 degrees in order to easily insert the injector into the skin, the end of the optical fiber fixed to the injector may be easily broken and the form of the oscillated laser beam is not accurately round and distorted in a round oval shape.

Meanwhile, acupuncture treatment is performed for the treatment acceptor in accordance with the reinforcing and reducing treatment method used in the science of acupuncture in order to maximize the effect of the acupuncture. At this time, when a relevant meridian pathway is weak, reinforcing treatment is performed while the relevant meridian pathway is strong, reducing treatment is performed. In a reinforcing and reducing treatment method by twirling and twisting the acupuncture of reinforcing and reducing treatment method, twirling the acupuncture left after injection is referred to as a reinforcing treatment method and twirling the acupuncture right after injection is referred to as a reducing treatment method. In a color treatment field, among lasers having various wavelengths, a red-based laser of 630 to 690 nm has the effect of the reinforcing treatment method and a green-based laser of 530 to 555 nm has the effect of the reducing treatment method. The semiconductor laser having various wavelengths is currently being commercialized and also has various powers, such that it is possible to effectively treat the affected parts by using the semiconductor laser having various wavelengths and powers that are suitable for treating diseases.

### SUMMARY OF THE INVENTION

In order to solve the above-mentioned problems, a first object of the present invention is to provide invasive laser acupuncture that can independently control two semiconductor lasers in a continuous mode and a pulse mode so as to adopt a reinforcing and reducing treatment method.

Further, a second object of the present invention is to provide invasive laser acupuncture using a metal-coated optical fiber acupuncture in which a reinforcing and reducing treatment method can be adopted.

In order to achieve the object of the present invention, an invasive laser acupuncture according to an aspect of the present invention includes: a first semiconductor laser connected to a first optical fiber acupuncture and providing a red-based laser beam with the first optical fiber acupuncture; a second semiconductor laser connected to a second optical fiber acupuncture and providing a green-based laser beam with the second optical fiber acupuncture; and a driving circuit independently driving the first semiconductor laser and the second semiconductor laser in a continuous mode or a pulse mode by a switching operation.

The driving circuit may include: a first static current supply unit that is connected to the first semiconductor laser, receives first voltage and supplies first static current to the first semiconductor laser, and turns on the first semiconductor laser; a second static current supply unit that is connected to the second semiconductor laser, receives the first voltage and supplies second static current to the second semiconductor laser, and turns on the second semiconductor laser; and a function generator that generates a sine wave having a predetermined frequency by receiving the second voltage and supplies the sine wave to the first and second static current supply units in the pulse driving.

The driving circuit may further include: a first continuous/pulse driving selection switch that controls to supply the first voltage to the first static current supply unit in the continuous mode driving and supply the second voltage to the function generator in the pulse mode driving; and a second continuous/pulse driving selection switch that controls to supply the first voltage to the second static current supply unit in the continuous mode driving and supply the second voltage to the function generator in the pulse mode driving.

The driving circuit may further include: a first amplifier that amplifies the sine wave which is an output of the function generator and supplies the amplified sine wave to the first static current supply unit; and a second amplifier that amplifies the sine wave which is the output of the function generator and supplies the amplified sine wave to the second static current supply unit.

A pulse interval or a peak value of the red or green laser beam may be adjusted in the pulse driving by adjusting the function generator with an external potentiometer.

At least one of the pulse peak value and a pulse width of the red or green laser beam can be adjusted by adjusting direct current of the first static current supply unit and the second static current supply unit.

At least one of the first optical fiber acupuncture and the second optical fiber acupuncture may include: a core; a cladding that clothes the core; and a metal coating layer that coats the exterior of the cladding with a metal having a predetermined thickness.

At least one of the first optical fiber acupuncture and the second optical fiber acupuncture may be manufactured by cutting an optical fiber acupuncture of a first length and removing a jacket of the optical fiber acupuncture, removing polymer materials that clothes the cladding of the optical fiber acupuncture without the jacket, and coating the exterior of the optical fiber acupuncture without the polymer materials with the metal having the predetermined thickness.

The end of at least one of the first optical fiber acupuncture and the second optical fiber acupuncture may be processed at an angle of 10 to 30 degrees.

According to an embodiment of the present invention, since invasive laser acupuncture can independently drive a red laser and a green laser in a continuous mode and a pulse mode, respectively, the invasive laser acupuncture can easily be adopted in a reinforcing and reducing treatment method in traditional oriental medicine and more effectively treat treatment acceptors.

Further, since each semiconductor laser can independently driven in the continuous mode or pulse mode, each semiconductor laser can operate so as to acquire the maximum power and since a laser driving circuit has a static current supply circuit for each semiconductor laser, it is possible to ensure the life-span of each semiconductor laser.

In addition, by using a metal-coated optical fiber acupuncture, an optical fiber acupuncture is probed into meridian pathways provided under an epidermal layer, such that it is possible to efficiently transmit a laser beam without loss.

Accordingly, since an invasive dual-wavelength laser acupuncture treatment apparatus according to an embodiment of the present invention can naturally substitute the effect of a metal acupuncture which is currently being operated in the traditional oriental medicine and can easily adopt the principle of a reinforcing and reducing treatment method which is one technique of the science of acupuncture, the invasive dual-wavelength laser acupuncture treatment can efficiently adopted in various disease treatment for the treatment acceptors depending on the type of a disease.

### BRIEF DESCRIPTION OF THE DRAWINGS

FIG. 1 is a block diagram illustrating known laser acupuncture using a He—Ne laser;

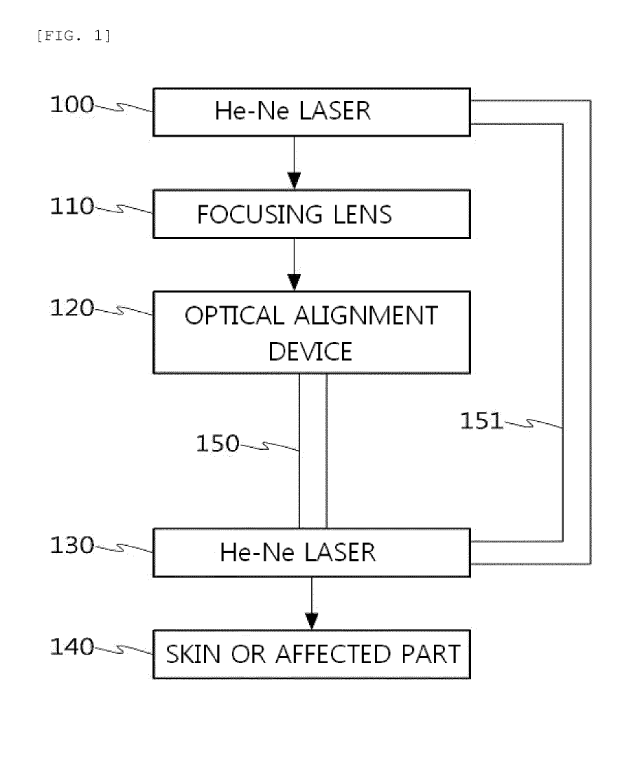

FIG. 2 is a block diagram illustrating known laser acupuncture using a semiconductor laser;

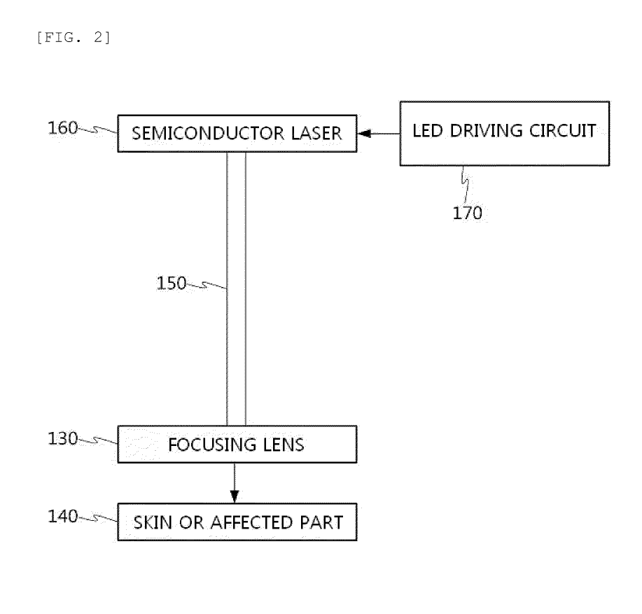

FIG. 3 is a circuit diagram illustrating a known driving circuit for driving a semiconductor laser;

FIG. 4 is a block diagram illustrating a configuration of invasive dual-wavelength laser acupuncture for operating a reinforcing and reducing treatment method according to an embodiment of the present invention;

FIG. 5 is a schematic diagram of an optical fiber acupuncture for optical transmission according to an embodiment of the present invention;

FIG. 6 is a partially enlarged cross-sectional view of part A of FIG. 5;

FIG. 7 is a partially enlarged cross-sectional view of part B of FIG. 5;

FIG. 8 illustrates a side photograph of an optical fiber acupuncture for optical transmission according to an embodiment of the present invention;

FIG. 9 illustrates a cross-sectional photograph of an optical fiber acupuncture for optical transmission according to an embodiment of the present invention;

FIG. 10 is a graph illustrating a current-output characteristic of a laser oscillated in a continuous mode of a red laser acupuncture according to an embodiment of the present invention;

FIG. 11 is a graph illustrating a current-output characteristic of a laser oscillated in a continuous mode of a green laser acupuncture according to an embodiment of the present invention;

FIG. 12 is a graph illustrating a pulse characteristic of a laser oscillated in a pulse mode of a red laser acupuncture according to an embodiment of the present invention; and

FIG. 13 is a graph illustrating a pulse characteristic of a laser oscillated in a pulse mode of a green laser acupuncture according to an embodiment of the present invention.

### DETAILED DESCRIPTION OF THE PREFERRED EMBODIMENTS

The present invention can make alternations and have various embodiments. Therefore, the embodiments are illustrated in the accompanying drawings and described in detail in the detailed description. However, the present invention is not limited to a predetermined embodiments and it should be understood that the present invention includes all alternations, equivalents or substitutions that are included in the spirit and scope of the present invention. Like elements refer to like reference numerals in describing the accompanying drawings.

Terms such as “first”, “second”. “A”, “B”, etc. may be used in describing various constituent elements, but the constituent elements should not be limited by the terms. The terms are used only for differentiate one constituent element from other constituent elements. For example, a first constituent element may be referred to as a second constituent element without departing from the scope of the appended claims and similarly, the second constituent element may also be referred to as the first constituent element. Terms such as “and/or” include a combination of a plurality of relevant disclosed items or any one of the plurality of relevant disclosed items.

When it is described that one constituent element is “joined” or “connected” to another constituent element, one constituent element may be joined or connected directly to another constituent element, but it will be appreciated that a third constituent element may be provided therebetween. On the contrary, when it is described that one constituent element is “directly joined” or “directly connected” to another constituent element, it will be appreciated that no constituent element is provided therebetween.

Terms used in this application are used for just describing predetermined embodiments and not used for limiting the present invention. Expression of the singular number includes expression of the plural numbers if the singular number does not have a meaning different from the plural numbers. In this application, it will be appreciated that terms “include” or “have” are used for indicating that characteristics, numbers, steps, operations, constituent elements, components or combinations thereof are provided and existence or adding possibility of one or more different characteristics or numbers, steps, operations, constituent elements, components, and combinations thereof is not previously excluded.

If not differently defined, all terms disclosed herein including technical or scientific terms have the same meanings as those generally by those skilled in the art. It should be understood that generally used terms that are defined in dictionaries have meanings that coincide with contextual meanings of relevant technologies and if definitely defined in this application, the terms should not be interpreted as ideal or excessively formal meanings.

Hereinafter, preferred embodiments of the present invention will be described in detail with reference to the accompanying drawings.

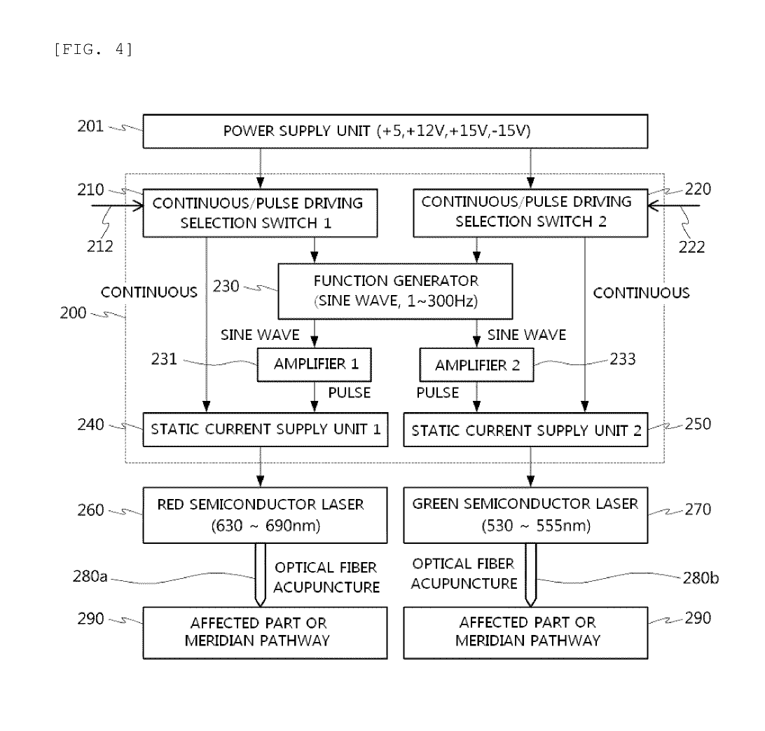

FIG. 4 is a block diagram illustrating a configuration of invasive dual-wavelength laser acupuncture for operating a reinforcing and reducing treatment method according to an embodiment of the present invention.

Referring to FIG. 4, the laser acupuncture according to the embodiment of the present invention includes a driving circuit 200, a red semiconductor laser 260, a green semiconductor laser 270, and optical fiber acupunctures 280a and 280b. The laser acupuncture according to the embodiment of the present invention may further include a power supply unit 201. The power supply unit 201 may be implemented in the laser acupuncture or implemented outside separately from the laser acupuncture. The power supply unit 201 may be implemented to include the driving circuit 200 therein or implemented outside of the driving circuit 200.

The driving circuit 200 may include a first continuous/pulse driving selection switch 210, a second continuous/pulse driving selection switch 220, a function generator 230, a first static current supply unit 240, and a second static current supply unit 250.

The laser acupuncture according to the embodiment of the present invention generates a laser beam by independently driving the red-based semiconductor laser 260 and the green-based semiconductor laser 270. The red-based semiconductor laser 260 generates a red-based laser beam having a first wavelength which is one of 635 nm, 650 nm, nm, 655 nm, 658 nm, 660 nm, 670 nm, 685 nm, and 690 nm. Preferably, the red-based semiconductor laser 260 can generate the red-based laser beam having the first wavelength which is one of 635 nm, 650 nm, 654 nm, 655 nm, nm, 660 nm, and 670 nm which belong to approximately nm to 670 nm, which are reported to have a reinforcing treatment effect. Preferably, the green-based semiconductor laser 270 can generate a green-based laser beam having a second wavelength of 532 nm that belongs to approximately 530 nm to 555 nm that are reported to have the reinforcing treatment effect. Two semiconductor lasers selected to adopt a reinforcing and reducing treatment method of the present invention can use a red laser having the wavelength of 658 nm, for example and a green laser having the wavelength of 532 nm, for example.

The power supply unit 201 may have outputs of, for example, +5 Volt, +12 Volt, and ±15 Volt for continuous driving and pulse driving of the semiconductor laser. Herein, the power supply unit 201 can drive, with the voltage of +5 V, the first static current supply unit 240 for driving the red semiconductor and the second static current supply unit 250 required to operate the green semiconductor laser. The power supply unit 201 can drive a function generator 230 with the voltage of +12 V. Further, the power supply unit 201 may drive other electronic elements such as amplifiers 231 and 233 with the voltage of +15 V. Of course, the power supply unit 201 may generate the voltages of +5 Volt, +12 Volt, and ±15 Volt with one power supply circuit chip or generate the voltages of +5 Volt and ±15 Volt with one power supply circuit chip and generate the voltage of +12 Volt with the other power supply circuit chip or generate the voltages of +5 Volt, +12 Volt, and ±15 Volt with power supply circuit chips, respectively.

The first static current supply unit 240 is connected to the red semiconductor laser 260 and turns on the red semiconductor laser 260 by providing the first static current to the red semiconductor laser 260 and receiving first voltage of +5 V depending on a switching operation of the first continuous/pulse driving selection switch 210.

The second static current supply unit 250 is connected to the green semiconductor laser 270 and turns on the green semiconductor laser 270 by providing the second static current to the green semiconductor laser 270 and receiving the first voltage of +5 V depending on a switching operation of the second continuous/pulse driving selection switch 220.

The function generator 230 receives second voltage of 2 V and generates a sine wave of a predetermined frequency to supply the voltage of 2 V to the first and second static current supply units 240 and 250 at the time of driving the pulse mode depending on the switching operation of the first and second continuous/pulse driving selection switches 210 and 220.

The first continuous/pulse driving selection switch 210 is controlled to supply the first voltage to the first static current supply unit 240 in driving the continuous mode and supply the second voltage to the function generator 230 in driving the pulse mode.

The second continuous/pulse driving selection switch 220 is controlled to supply the first voltage to the second static current supply unit 250 in driving the continuous mode and supply the second voltage to the function generator 230 in driving the pulse mode.

The first amplifier 231 amplifies and supplies the sine wave which is an output of the function generator 230 to the first static current supply unit 240.

The second amplifier 233 amplifies and supplies the sine wave which is the output of the function generator 230 to the second static current supply unit 250.

The first and second continuous/pulse driving selection switches 210 and 220 can be implemented by dial switches.

The first continuous/pulse driving selection switch 210 is selected in the continuous mode in response to a first control signal 212 for continuous oscillation of the red semiconductor laser 260. Herein, the first control signal 212 may be generated when a user manually turns the dial switch or may be generated in hardware by a separate control unit (not shown) or in software by programming. At this time, the first static current supply unit 240 is operated by the power supply unit 201 and for example, the red semiconductor laser 260 having a wavelength of 658 nm is turned on to be continuously oscillated. Herein, the power of the red semiconductor laser 260 can easily be adjusted by a potentiometer that is connected to the outside. The continuously oscillated red laser beam is transmitted to an affected part or meridian pathways 290 through the metal-coated optical fiber acupuncture 280a without loss according to the embodiment of the present invention.

The optical fiber acupunctures 280a and 280b according to the embodiment of the present invention is contrived to serve as optical transmission and a metal acupuncture.

In the same manner as above, the second continuous/pulse driving selection switch 220 is selected in the continuous mode in response to a second control signal 222 for continuous oscillation of the green semiconductor laser 270. The second control signal 222 may be generated when the user manually turns the dial switch and or may be generated in hardware by the separate control unit (not shown) or in software by programming. At this time, the second static current supply unit 250 is operated by the power supply unit 201 and for example, the green semiconductor laser 270 having a wavelength of 532 nm is turned on to be continuously oscillated. The continuously oscillated green laser beam is transmitted to the affected part or the meridian pathways 290 through the metal-coated optical fiber acupuncture 280b.

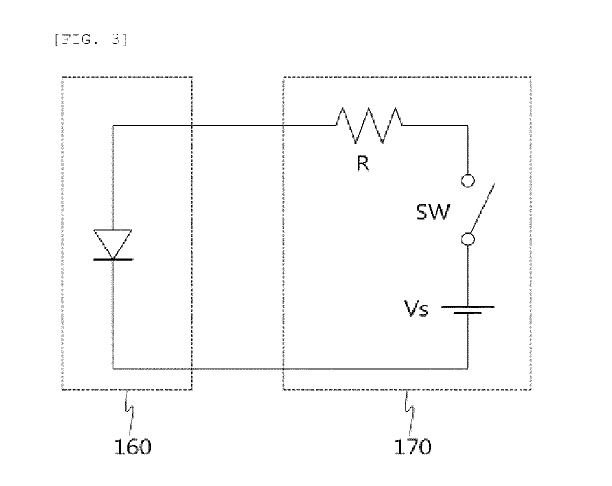

Herein, unlike the known laser acupuncture driving device, since the red semiconductor laser 260 and the green semiconductor laser 270 are independently controlled by the first static current supply unit 240 and the second static current supply unit 250, the laser acupuncture according to the embodiment of the present invention can be operated regardless of turning on-off one of the two lasers and can be oscillated at the maximum power of each laser. Further, by sublating a scheme of turning on the semiconductor laser with the LED driving circuit 170 connected to one resistor of the known laser acupuncture driving device of FIG. 3 in series and manufacturing the separate static current supply units 240 and 250, since an external potentiometer is adjusted and the power of the laser can be controlled only by increasing and decreasing direct current, a problem in lifespan reduction of the laser in the known laser acupuncture driving device can be solved.

In the case of the pulse driving, first, the first continuous/pulse driving selection switch 210 is selected as the pulse mode in response to the first control signal 212 for pulse oscillation of the red semiconductor laser 260. Therefore, the function generator 230 is operated by the power supply unit 201, such that the sine wave having a predetermined frequency is supplied to the first static current supply unit 240. The frequency of the sine wave oscillated from the function generator 230 varies from 1 Hz to 300 Hz, for example and a peak value of the sine wave can be controlled from −10 V to +10 V, for example. At this time, the frequency and the peak value of the sine wave can be controlled by the potentiometer connected to the outside. The sine wave inputted into the first static current supply unit 240 is converted to a current pulse for turning on the red semiconductor laser 260 having the wavelength of 658 nm through the first static current supply unit 240 and the red semiconductor laser 260 oscillates with the corresponding peak value, pulse width, and pulse repetition rate by the current pulse. In the same manner as the continuous oscillation, the pulse-oscillated red laser beam is transmitted to the affected part or the meridian pathways 290 through the metal-coated optical fiber 280a.

In the same manner as above, the second continuous/pulse driving selection switch 220 is selected as the pulse mode in response to the second control signal 222 for the pulse oscillation of the green semiconductor laser 270. Similarly as the red laser oscillation, the function generator 230 is operated by the power supply unit 201, such that the sine wave having the predetermined frequency is supplied to the second static current supply unit 250. The frequency of the sine wave oscillated from the function generator 230 varies from 1 Hz to 300 Hz, for example and the peak value of the sine wave can be controlled from −10 V to +10 V, for example. At this time, the frequency and the peak value of the sine wave can be controlled by the potentiometer connected to the outside. The sine wave is converted to a current pulse for turning on the green semiconductor laser 270 having the wavelength of 532 nm through the second static current supply unit 250 and the green semiconductor laser 270 oscillates with the corresponding peak value, pulse width, and pulse repetition rate by the current pulse. Therefore, the pulse-oscillated green laser beam is transmitted to the affected or the meridian pathways 290 through the metal-coated optical fiber acupuncture 280b.

The invasive dual-wavelength acupuncture treatment apparatus for operating the reinforcing and reducing treatment method according to the embodiment of the present invention can be connected to the red semiconductor laser 260 having the wavelength of 658 nm and the green semiconductor laser 270 having the wavelength of 532 nm in which a multimode optical fiber having a core diameter of 50 μm is pigtailed, for example.

As described above, most of the known laser acupuncture treatment apparatuses just irradiate a laser beam focused in a non-invasive method onto a skin or affected part. However, reflection or scattering of the laser beam on the surface of the skin interferes with effectively transmitting laser energy to the meridian pathways. In order to complement the disadvantage, the laser beam may be irradiated by inserting and fixing the optical fiber into an intravenous injector and injecting the intravenous injector, but the end of the optical fiber becomes contaminated and frequently broken due to brittleness of the optical fiber. In particular, when the end of the optical fiber is damaged during the injection or after the injection, the irradiated laser beam is distorted, such that the power of the laser beam is remarkably reduced and small glass pieces of the optical fiber remains in a human body to be seriously hazardous to the human body.

As a result, compared with the known laser acupuncture treatment apparatus, the invasive dual-wavelength laser acupuncture according to the embodiment of the present invention can substitute for the known metal acupuncture, is resistant to the brittleness, and uses the metal-coated optical fiber acupuncture which can efficiently transmit light.

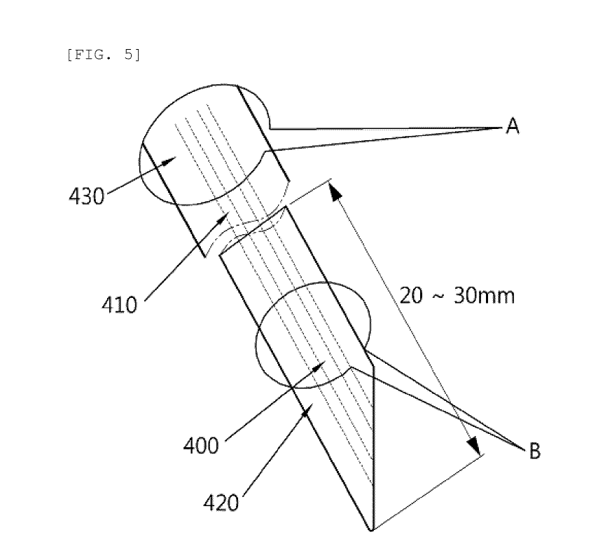

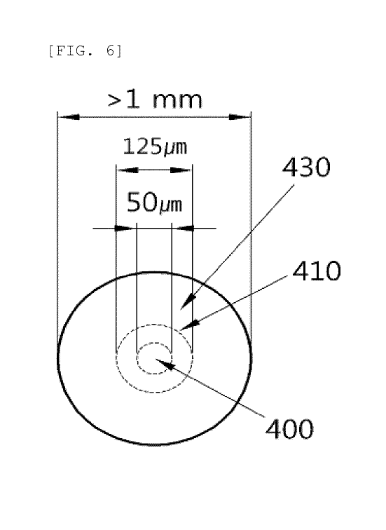

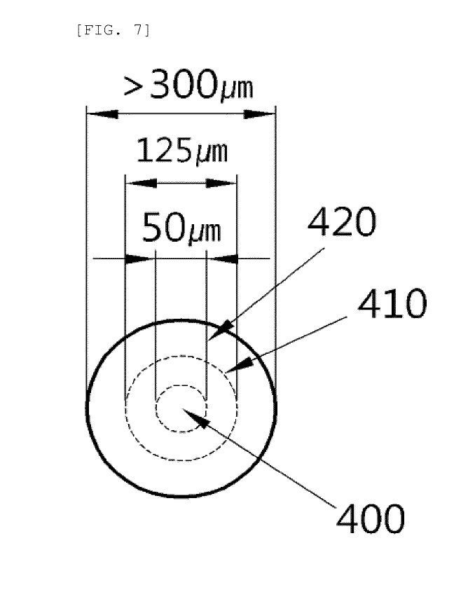

FIG. 5 is a schematic diagram of an optical fiber acupuncture for optical transmission, which is used in the laser acupuncture according to an embodiment of the present invention. FIG. 6 is a partially enlarged cross-sectional view of part A of FIG. 5 and FIG. 7 is a partially enlarged cross-sectional view of part B of FIG. 5.

As shown in FIG. 6, a general multimode optical fiber for optical transmission has a diameter of a core 400 of 50 μm or more and a diameter of a cladding 410 of 125 μm to cause internal total reflection of the laser beam in the optical fiber by surrounding the core 400. Further, in the case of the general multimode optical fiber for the optical transmission, the exterior of the cladding 410 is polymer-coated in order to protect the optical fiber from external impact and finally, the exterior of the cladding 410 is covered with a jacket 430 having a thickness of 1 mm or more.

In order to manufacture the metal-coated optical fiber acupuncture according to the embodiment of the present invention, which has a length similar to the known metal acupuncture, approximately 20 to 30 nm of the jacket 430 is first removed as shown in FIG. 5.

By removing polymer materials clothing the cladding 410 with acetone or a stripper for the optical fiber, the metal-coated optical fiber acupuncture is configured to have a predetermined diameter, i.e., 300 μm or more as shown in FIG. 7. Since the metal acupuncture generally has a diameter of approximately 300 to 500 μm, a metal-coated optical fiber acupuncture having a diameter of 350 μm is manufactured with electroplating after thinly coating the exterior of the cladding 410 with gold in the embodiment of the present invention. The metal used in the electroplating includes titanium, gold, silver, nickel, or stainless steel and may include any metallic material when the metal is harmless to the human body except for heavy metals.

The end of the metal-coated optical fiber acupuncture is processed at a proper angle so that the metal-coated optical fiber acupuncture can easily be injected into the skin. In general, one side of the intravenous injector is processed at 10 to 30 degrees and the metal acupuncture is pointed at the end. A femtosecond laser may be used in order to process one side of the optical fiber acupuncture according to the embodiment of the present invention and femtosecond laser processing or hydrofluoric acid (HF) etching may be used in order to process the end of the optical fiber acupuncture to be pointed.

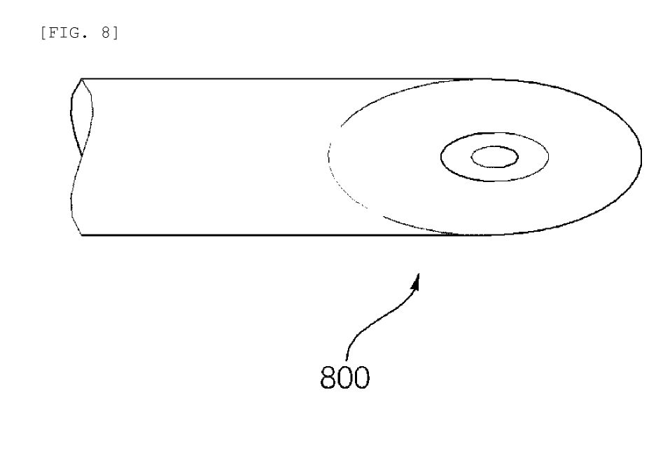

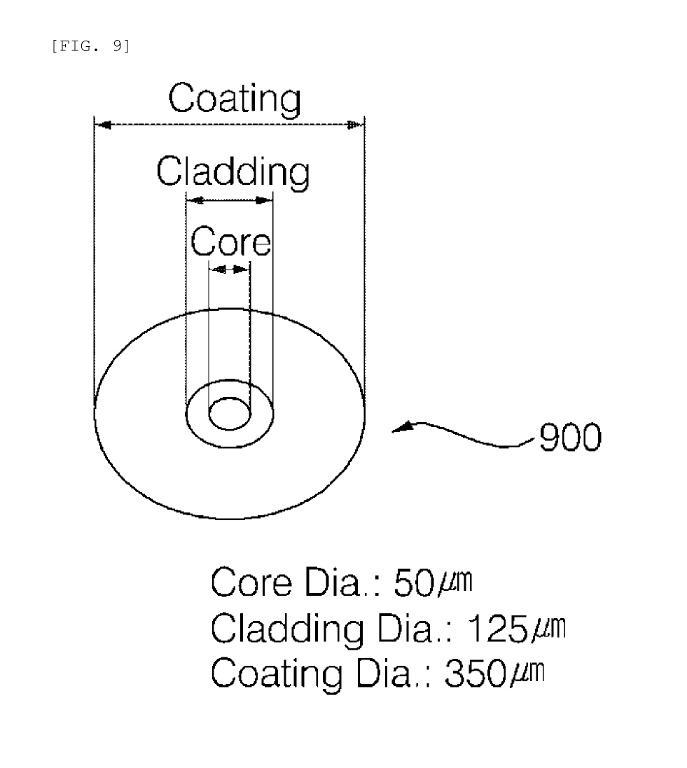

FIG. 8 illustrates a side photograph of an optical fiber acupuncture for optical transmission according to an embodiment of the present invention and FIG. 9 illustrates a cross-sectional photograph of an optical fiber acupuncture for optical transmission according to an embodiment of the present invention. As shown in FIG. 8, from the side photograph 800 of the optical fiber acupuncture, the optical fiber acupuncture is processed at approximately 20 degrees by using the femtosecond processing. As shown in FIG. 9, a diameter of the metal coating measured from the cross-sectional photograph 900 of the optical fiber acupuncture is set to 350 μm and can replace the know metal acupuncture in the traditional oriental medicine.

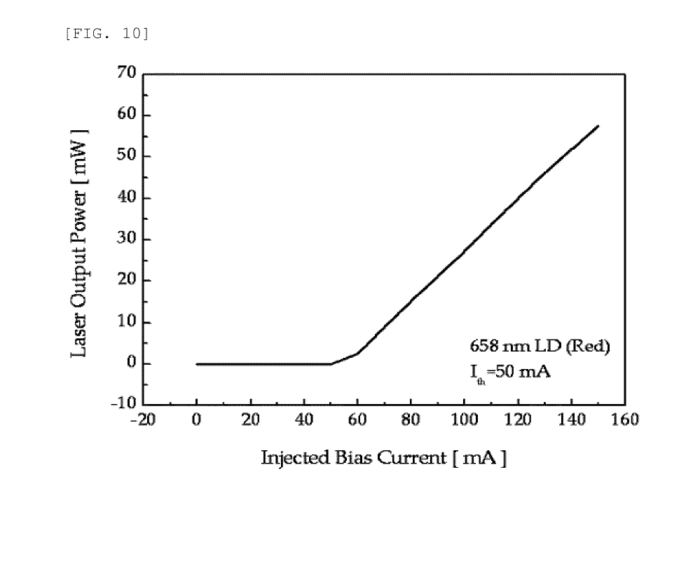

FIG. 10 is a graph illustrating a current-output characteristic of a laser oscillated in a continuous mode of a red laser acupuncture according to an embodiment of the present invention.

Referring to FIG. 10, the characteristic of the current I—laser power P (I-P graph) can be shown when the red semiconductor laser according to the embodiment of the present invention operates in the continuous oscillation mode. The I-P graph illustrates the laser power to the applied current which is the most primary characteristic of the characteristics of the semiconductor laser. As shown in FIG. 10, threshold current at which a red laser beam having the wavelength of 658 nm starts to oscillate is approximately 50 mA and the power of the red laser has a linear characteristic with respect to current of 50 mA or more. In other words, the semiconductor laser beam oscillated through the static current supply unit 240 according to the embodiment of the present invention is stably outputted in proportion to increased and decreased current. The red semiconductor laser having the wavelength of 658 nm, which is used in the embodiment of the present invention has the maximum power of 60 mW with respect to the maximum applied current of 150 mA.

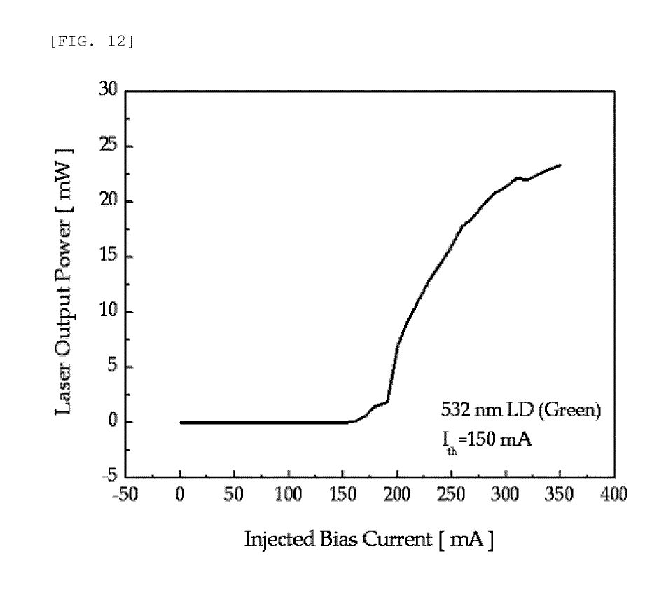

FIG. 12 is a graph illustrating a pulse characteristic of a laser oscillated in a pulse mode of a red laser acupuncture according to an embodiment of the present invention.

As specifically described in FIG. 4 above, the pulse repetition rate, pulse width, and pulse peak value of the laser acupuncture according to the embodiment of the present invention can easily be adjusted by the external potentiometer in operating the pulse mode. The pulse repetition rate can be controlled from 1 Hz to 300 Hz. In order to improve precision in the frequency band of 1 Hz to less than 30 Hz primarily used in the traditional oriental medicine, a nonlinear potentiometer having high sensitivity in a low-frequency band may be used.

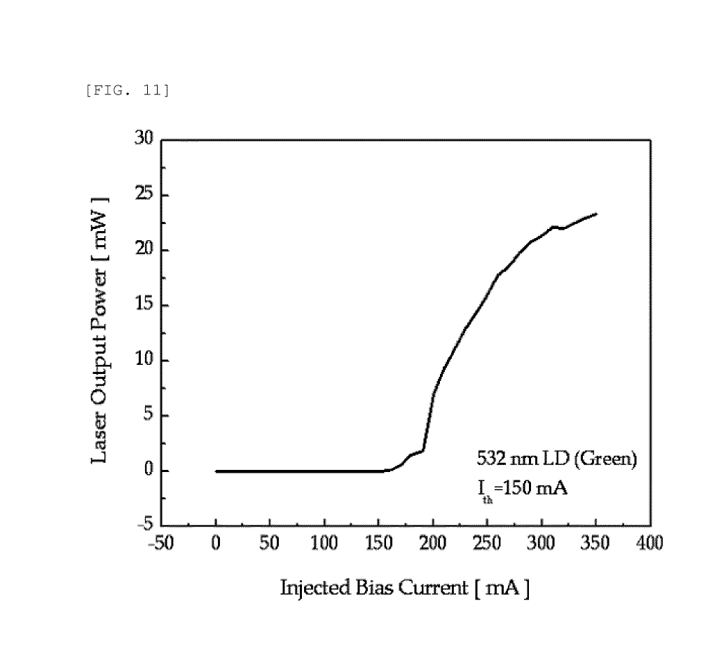

FIG. 11 is a graph illustrating a current-output characteristic of a laser oscillated in a continuous mode of a green laser acupuncture according to an embodiment of the present invention.

Referring to FIG. 11, threshold current at which the green laser beam having a wavelength of 532 nm starts to be oscillated is approximately 150 mA and the power of the red laser has a substantially linear characteristic with respect to current of 150 mA or more, but the power characteristic of the green semiconductor laser is a bit inferior to the power characteristic of the red semiconductor laser. The green semiconductor laser having the wavelength of 532 nm, which is used in the embodiment of the present invention has the maximum power of 30 mW with respect to the maximum applied current of 600 mA.

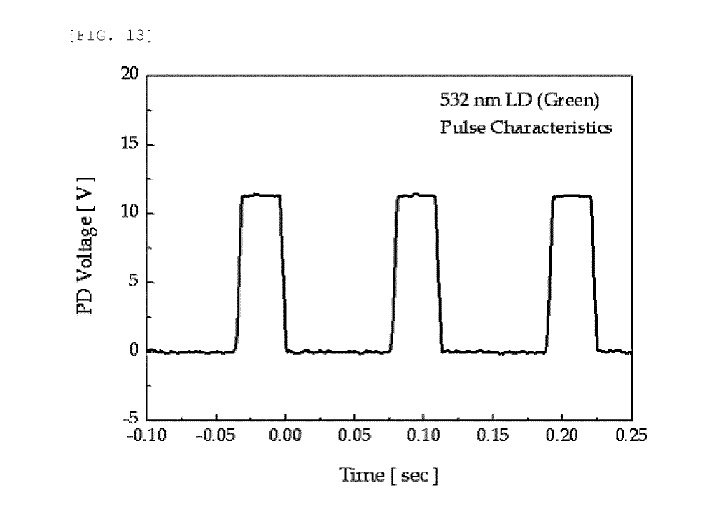

FIG. 13 is a graph illustrating a pulse characteristic of a laser oscillated in a pulse mode of a green laser acupuncture according to an embodiment of the present invention.

Referring to FIG. 3, in the same manner as the red semiconductor laser, the green semiconductor laser is controlled and has an oscillation characteristic similar to the pulse oscillation of the red laser of FIG. 12. The pulse repetition rate can be controlled from 1 Hz to 300 Hz and may be controlled from 1 Hz to 30 Hz which is the frequency band primarily used in the traditional oriental medicine by using the nonlinear potentiometer having the high sensitivity in the low-frequency band.

According to the embodiment of the present invention, the laser beam is independently driven in the continuous and pulse modes, respectively, by using the semiconductor laser having a red wavelength of 630 to 690 nm and a green wavelength of 530 to 555 nm to maximize a treatment effect depending on the type of diseases and the reinforcing and reducing treatment method and arbitrarily adjust the peak value, pulse width, and pulse repetition rate in the pulse mode. Further, in order to overcome the problem of the non-invasive laser acupuncture having reflection or scattering loss on the surface of the skin, the metal-coated optical fiber replaces the metal acupuncture, thereby maximizing laser-ray treatment.

Further, according to the embodiment of the present invention, the laser beam can be oscillated in the continuous and pulse modes by using electronic devices including the function generator, amplifier, static current supply unit, etc. and the low-power semiconductor laser connected to the optical fiber acupuncture and the laser beams having two different red and green wavelengths are independently controlled and transferred to the affected parts of the treatment acceptors through the optical fiber, such that the effective reinforcing and reducing treatment method can be adopted, thereby maximizing the effect of the traditional oriental treatment.

Further, according to the embodiment of the present invention, the invasive optical fiber acupuncture using the metal-coated optical fiber probe is provided to have an injection effect corresponding to the metal acupuncture and since irradiated light is just transferred to the meridian pathways without causing any loss, traditional oriental medical apparatuses which can maximize the effect of the laser-ray treatment can be provided in spite of using the low-power semiconductor laser as a light source.

Further, according to the embodiment of the present invention, since the separate static current supply unit is used when the semiconductor laser operates in the pulse mode without the known LED driving circuit, a long life-span of the laser can be ensured and since the pulse repetition rate is easily adjusted by the function generator and the pulse width and the peak pulse value is easily adjusted depending on external voltage, the invasive dual-wavelength laser acupuncture treatment apparatus can be provided, which allows an operator to arbitrarily change desired power and laser wavelength depending on the type of diseases and operated parts.

Although preferred embodiments of the present invention have been described, it will be appreciated by those skilled in the art that various modifications and change can be made without departing from the spirits and scope of the appended claims of the present invention.

What is claimed is:

1. An invasive laser acupuncture, comprising:
a first semiconductor laser connected to a first optical fiber acupuncture and providing red-based laser beam with the first optical fiber acupuncture;
a second semiconductor laser connected to a second optical fiber acupuncture and providing a green-based laser beam with the second optical fiber acupuncture; and
a driving circuit independently driving the first semiconductor laser and the second semiconductor laser in a continuous mode or a pulse mode by a switching operation.

2. The invasive laser acupuncture according to claim 1, wherein the driving circuit includes:
a first static current supply unit that is connected to the first semiconductor laser, receives first voltage and supplies first static current to the first semiconductor laser, and turns on the first semiconductor laser;
a second static current supply unit that is connected to the second semiconductor laser, receives the first voltage and supplies second static current to the second semiconductor laser, and turns on the second semiconductor laser; and
a function generator that generates a sine wave having a predetermined frequency by receiving the second voltage and supplies the sine wave to the first and second static current supply units in the pulse driving.

3. The invasive laser acupuncture according to claim 2, wherein driving circuit includes:
a first continuous/pulse driving selection switch that controls to supply the first voltage to the first static current supply unit in the continuous mode driving and supply the second voltage to the function generator in the pulse mode driving; and
a second continuous/pulse driving selection switch that controls to supply the first voltage to the second static current supply unit in the continuous mode driving and supply the second voltage to the function generator in the pulse mode driving.

4. The invasive laser acupuncture according to claim 3, wherein the driving circuit further includes:
a first amplifier that amplifies the sine wave which is an output of the function generator and supplies the amplified sine wave to the first static current supply unit; and
a second amplifier that amplifies the sine wave which is the output of the function generator and supplies the amplified sine wave to the second static current supply unit.

5. The invasive laser acupuncture according to claim 2, wherein a pulse interval or a peak value of the red or green laser beam is adjusted in the pulse driving by adjusting the function generator with an external potentiometer.

6. The invasive laser acupuncture according to claim 2, wherein at least one of the pulse peak value and a pulse width of the red or green laser beam is adjusted by adjusting direct current of the first static current supply unit and the second static current supply unit.

7. The invasive laser acupuncture according to claim 1, wherein at least one of the first optical fiber acupuncture and the second optical fiber acupuncture includes:
a core;
a cladding that clothes the core; and
a metal coating layer that coats the exterior of the cladding with a metal having a predetermined thickness.

8. The invasive laser acupuncture according to claim 7, wherein at least one of the first optical fiber acupuncture and the second optical fiber acupuncture is manufactured by cutting an optical fiber acupuncture by a first length and removing a jacket of the optical fiber acupuncture, removing polymer materials that clothes the cladding of the optical fiber acupuncture without the jacket, and coating the exterior of the cladding of the optical fiber acupuncture without the polymer materials with the metal having the predetermined thickness.

9. The invasive laser acupuncture according to claim 7, wherein the end of at least one of the first optical fiber acupuncture and the second optical fiber acupuncture is processed at an angle of 10 to 30 degrees.
